(19) United States
(12) Patent Application Publication
(10) Pub. No.: US 2024/0409593 A1
(43) Pub. Date: Dec. 12, 2024

(54) COMPOSITION FOR SUPPRESSION OF INFLAMMATORY DISEASES COMPRISING BIOCOMPATIBLE POLYPEPTIDE TO WHICH FUNCTIONAL PEPTIDE IS CONJUGATED

(71) Applicant: BIOVIT CO., LTD., Seoul (KR)

(72) Inventors: Yong Tae KIM, Gyeonggi-do (KR); Eun Bin CHU, Seoul (KR)

(21) Appl. No.: 18/039,333

(22) PCT Filed: Oct. 15, 2021
(86) PCT No.: PCT/KR2021/014295

§ 371 Date: May 30, 2023

(30) Foreign Application Priority Data
Nov. 30, 2020 (KR) 10-2020-0163705

(51) Int. Cl. C07K 14/435; A61K 8/64; A61K 38/00; A61K 47/64; A61P 29/00; A61Q 19/00

(52) U.S. Cl. CPC C07K 14/43509; A61K 8/64; A61K 47/64; A61P 29/00; A61Q 19/00; A61K 38/00

(57) ABSTRACT

The present invention relates to a composition for the suppression or amelioration of inflammatory diseases, comprising a recombinant polypeptide in which a functional peptide is conjugated to a biocompatible polypeptide. The composition of the present invention is not only safe for the human body, but can also be widely applied to the suppression of inflammation by inhibiting cytokines and tumor necrosis factors that cause early inflammation. Thus, the composition can be used in fields such as medicines, medical products, cosmetics, and daily necessities, to suppress inflammation, and can also be used in the development of additional new drugs.

### CROSS REFERENCE TO RELATED APPLICATIONS

This is a national stage application filed under 37 U.S.C. 371 based on International Patent Application No. PCT/KR2021/014295, filed Oct. 15, 2021, which claims the benefit of and priority to Korean Patent Application No. 10-2020-0163705, filed on Nov. 30, 2020, the disclosures of each are incorporated herein by reference in their entireties.

### SEQUENCE LISTING

The application contains a Sequence Listing which has been submitted electronically in .txt format together with the International Application and is hereby incorporated by reference in its entirety. Said .txt copy, created on Oct. 2, 2023, is named “2020OPA4536PCUS-SL.txt” and is 29,147 bytes in size. The sequence listing contained in this .txt file is part of the specification and is hereby incorporated by reference herein in its entirety.

### TECHNICAL FIELD

The present invention relates to a composition for suppressing inflammatory diseases, comprising a biocompatible polypeptide to which a functional peptide is conjugated, and specifically, to a composition for suppressing various inflammatory actions, comprising a recombinant polypeptide in which a functional peptide is conjugated to a mussel adhesive protein-derived biocompatible polypeptide, for example, medicines, medical products, cosmetics, daily necessities, and the like.

### BACKGROUND ART

Inflammation is one of the defense responses of biological tissues to stimuli, and immune cells, blood vessels, inflammatory mediators, and the like are involved, and all factors that cause cell damage can contribute to inflammation. The inflammatory response is the most important biological defense mechanism of the human body that restores physiological functions in vivo by removing factors of damage applied to the body and regenerating damaged tissues.

The human inflammatory response is regulated through several complex systems. When an inflammatory response occurs, blood flow is increased, blood capillary permeability is improved, migration of large molecules such as leukocytes, antibodies, cytokines and complements to sites of injury, infection or immune response is induced, and high blood flow and plasma leakage to the site of inflammation cause heat, redness, swelling, and pain (Calder PC. n-3 Polyunsaturated fatty acids, inflammation, and inflammatory diseases. Am J Clin Nutr. 2006; 83 (6): 1505S-19S). In addition, the inflammatory response induced by inflammatory stimuli such as pathogen-derived molecules, products of damaged cells, toxins or allergens leads to the release of some cell-derived mediators, such as interleukin (IL) family cytokines and tumor necrosis factor (TNF).

Recently, the incidence of hypersensitive or inflammatory immune diseases is rapidly increasing along with changes in the living environment due to industrialization and westernization, and when inflammation becomes chronic, it may appear not only as autoimmune diseases such as atopy and rheumatoid arthritis, but also as diseases such as adult diseases, cardiovascular diseases and cancer.

However, in despite of causing such diseases, the inflammatory response has a very complex mechanism, and its mechanism of action has only been partially elucidated, and most drugs for treating inflammation are only used incidentally or empirically. Typically, non-steroidal substances used to suppress inflammation are ibuprofen and indomethacin, and the like, and steroidal substances are dexamethasone, and the like, but even in these cases, it is only recently that detailed pharmacological actions have begun to be revealed, and their use is limited due to problems with human safety.

On the other hand, the adhesive secreted by mussels or barnacles is a bioadhesive composed of protein, has water resistance, and is an environmentally friendly adhesive that is biodegradable by the action of microorganisms or enzymes due to its inherent biodegradable properties. Among them, the mussel adhesive protein secreted from the byssus of the mussel is a bio-based material derived from living organisms and is known to not attack human cells or cause an immune response, and thus, has been attracting attention as a biomaterial with great potential for application in the medical field, such as the adhesion of living tissues during surgery or the adhesion of broken teeth (Korean Patent No. 10-1578522 and Korean Patent No. 10-1631704). In fact, in order to utilize the mussel adhesive protein as a biocompatible material, the biological properties have been confirmed. In order to confirm the effect of contact and administration of the mussel adhesive protein, researches on toxicity and immune response to organs were conducted through animal experiments, and as a result, no specific findings due to toxicity were found for each organ, and no tissue necrosis, acute inflammatory response, or the like was observed (J. Dove, et al., Journal of American Dental Association 112, 879, 1986). Accordingly, most of the existing patents and researches on mussel adhesive proteins have focused on adhesiveness, which is their main characteristic (Korean Laid-open Patent Publication No. 10-2018-0033517 and Korean Laid-open Patent Publication No. 10-2018-0029228).

Researches on mussel adhesive proteins are being divided into development of various mussel proteins and mass production using genetic recombination technology, and mussel adhesive proteins derived from Mytilus edulis, Mytilus galloprovincialis and Mytilus coruscus are mainly known. In order to reveal the mechanism of mussel adhesive protein, the types of foot proteins and their amino acid sequences have been studied for a long time, and it has been found to be composed of about 12 or more types of proteins.

On the other hand, antimicrobial peptides are known to be members of the natural immune system, to be one of the safest antimicrobial substances with excellent biocompatibility, and to regulate host defense functions as well as direct bactericidal action. By acting with a different mechanism of action from conventional antibiotics, it is known that they may exhibit a wide range of powerful killing activities including antibiotic-resistant bacteria as well as fungi, and the action time to control pathogenic microorganisms is also very fast (Korea Patent No. 10-1652263). However, the anti-inflammatory effect of antimicrobial peptides is not specifically known in the art.

Therefore, the present inventors have developed a recombinant polypeptide in which various types of antimicrobial peptides were fused to a biocompatible polypeptide, and have revealed that the recombinant polypeptide exhibited an anti-inflammatory effect more effectively and could be applied to inflammatory diseases such as atopy, and have completed the present invention.

### DISCLOSURE

### Technical Problem

The present invention is to solve the problems of the prior art as described above, and it is an object of the present invention to provide a pharmaceutical composition for preventing or treating inflammatory diseases, comprising a recombinant polypeptide in which a functional peptide is conjugated to a biocompatible polypeptide.

In addition, it is another object of the present invention to provide a cosmetic composition for ameliorating inflammatory diseases, comprising a recombinant polypeptide in which a functional peptide is conjugated to a biocompatible polypeptide.

### Technical Solution

In order to solve the above problems, the present invention provide a pharmaceutical composition for preventing or treating inflammatory diseases, comprising a recombinant polypeptide in which a functional peptide is conjugated to a biocompatible polypeptide.

In an embodiment, the inflammatory diseases may be, but are not limited to, inflammatory skin diseases, or inflammatory diseases of the stomach, esophagus, small intestine, urethra and conjunctiva. In a preferred embodiment, the inflammatory diseases may be, but are not limited to, atopic dermatitis, psoriasis, urticaria, hidradenitis suppurativa (HS), acne, asthma, contact dermatitis, rhinitis, conjunctivitis, arthritis, bronchitis, bedsores, ulcers, cancer, ichthyosis, pemphigus, acne, lupus erythematosus, skin aging, or skin wrinkles.

In an embodiment, the biocompatible polypeptide may be, but is not limited to, a mussel adhesive protein or a barnacle adhesive protein. In a preferred embodiment, the mussel may be, but is not limited to, Mytilus edulis, Mytilus galloprovincialis, or Mytilus coruscus.

In an embodiment, the mussel adhesive protein may be a protein in which a second peptide selected from the group consisting of a foot protein-1 (FP-1) comprising the sequence of any one of SEQ ID NO: 1 to SEQ ID NO: 5, FP-2 comprising the sequence of SEQ ID NO: 6 and FP-4 comprising the sequence of SEQ ID NO: 11 is fused to the C-terminal, the N-terminal or both terminals of a first peptide selected from the group consisting of FP-3 comprising the sequence of any one of SEQ ID NO: 7 to SEQ ID NO: 10, FP-5 comprising the sequence of any one of SEQ ID NO: 12 to SEQ ID NO: 15 and FP-6 comprising the sequence of SEQ ID NO: 16.

In a preferred embodiment, the mussel adhesive protein may be selected from the group consisting of, but is not limited to: FP-151 (SEQ ID NO: 17 to SEQ ID NO: 21) in which FP-1 is fused to both terminals of FP-5; FP-131 (SEQ ID NO: 22) in which FP-1 is fused to both terminals of FP-3; FP-251 (SEQ ID NO: 23) in which FP-2 and FP-1 are fused to both terminals of FP-5; and FP-353 (SEQ ID NO: 17 to SEQ ID NO: 24) in which FP-3 is fused to both terminals of FP-5.

In an embodiment, the functional peptide may be, but is not limited to, an antimicrobial peptide, an extracellular matrix protein, or a growth factor, preferably an antimicrobial peptide.

In an embodiment, the biocompatible polypeptide and the functional peptide may be conjugated directly or may be conjugated through a peptide linker or non-peptide linker, but are not limited thereto.

In a preferred embodiment, the antimicrobial peptide may be, but is not limited to, KLWKKWAKKWLKLWKA (SEQ ID NO: 29), FALALKALKKL (SEQ ID NO: 30), ILRWPWWPWRRK (SEQ ID NO: 31), AKRHHGYKRKFH (SEQ ID NO: 32), KWKLFKKIGAVLKVL (SEQ ID NO: 33), LVKLVAGIKKFLKWK (SEQ ID NO: 34), IWSILAPLGTTLVKLVAGIGQQKRK (SEQ ID NO: 35), GIGAVLKVLTTGLPALISWI (SEQ ID NO: 36), SWLSKTAKKGAVLKVL (SEQ ID NO: 37), KKLFKKILKYL (SEQ ID NO: 38), GLKKLISWIKRAAQQG (SEQ ID NO: 39), GWLKKIGKKIERVGQHTRDATIQGLGIAQQAANVAATAR (SEQ ID NO: 40), LKKLAKLALAF (SEQ ID NO: 41), KLLLKLLKKLLKLLKKK (SEQ ID NO: 42), THRPPMWSPVWP (SEQ ID NO: 43), GWLKKIGKWKIFKK (SEQ ID NO: 44), ILPWKWPWWPWRR (SEQ ID NO: 45), RRWWCRC (SEQ ID NO: 46), KLAKLAKKLAKLAK (SEQ ID NO: 47), LKKLAKLALAFILRWPWWPWRRK (SEQ ID NO: 48), RLLRRLLRRLLRRLLRRLLR (SEQ ID NO: 49), FLKLLKKLAAKLF (SEQ ID NO: 50), WLKKIGKKIERVGQHTRDATIQGLGIAQQAANVAATAR (SEQ ID NO: 51), or GTNNWWQSPSIQN (SEQ ID NO: 52).

In addition, the present invention provides a cosmetic composition for ameliorating inflammatory diseases, comprising a recombinant polypeptide in which a functional peptide is conjugated to a biocompatible polypeptide.

In addition, the present invention provides a method of suppressing or ameliorating inflammatory diseases, comprising the step of: administering to a subject the recombinant polypeptide in which a functional peptide is conjugated to a biocompatible polypeptide, or a composition, for example, a pharmaceutical composition or a cosmetic composition, comprising the recombinant polypeptide.

### Advantageous Effects

The composition comprising the recombinant polypeptide of the present invention is not only safe for the human body, but also can be widely applied to suppress inflammation by inhibiting cytokines and tumor necrosis factors that cause early inflammation, and thus, it may be used in the fields of medicines, medical products, cosmetics, daily necessities, and the like for suppressing inflammation, and may also be used for the development of additional new drugs.

### DESCRIPTION OF DRAWINGS

The above described and other features and advantages of the present invention will become apparent when considering together the following embodiments and drawings, which are provided for illustrative purposes.

FIG. 1 shows a three-dimensional structural model of the recombinant polypeptide of the present invention. In the recombinant polypeptide, a functional peptide is conjugated to the N-terminal and C-terminal of the mussel adhesive protein. MAP represents mussel adhesive protein.

FIG. 2 is a graph showing that the inflammatory cytokine IL-6 is remarkably suppressed after treatment with a composition comprising the recombinant polypeptides BVI181, BVI182 and BVI183 of the present invention.

FIG. 3 is a graph showing that the tumor necrosis factor TNF-α is remarkably suppressed after treatment with a composition comprising the recombinant polypeptides BVI181, BVI182 and BVI183.

FIG. 4 is a graph showing that the ear thickness of mice treated with 12-O-tetradecanoylphorbol-13-acetate (TPA) is similar to that of a normal control group after treatment with a composition comprising the recombinant polypeptide BVI183, and thus, inflammation is reduced.

FIG. 5 is a result confirming that inflammation is remarkably reduced after treatment with a composition comprising the recombinant polypeptide BVI183 of the present invention in the inflamed dog skin.

### BEST MODE

Hereinafter, the present invention will be described in detail.

The present invention provides a composition for preventing or treating inflammatory diseases, comprising a recombinant polypeptide in which a functional peptide is conjugated to a biocompatible polypeptide.

In the present invention, the term “inflammatory disease” is used in the sense of including, without limitation, any disease caused by proinflammatory substances (inflammatory cytokines), such as TNF-α, IL-1, IL-6, prostaglandin, leukotriene and nitric oxide (NO), secreted from immune cells such as macrophages by excessively enhancing the human immune system due to harmful stimuli, such as proinflammatory factors.

In the present invention, the term “treatment”, unless otherwise stated, is used in the sense of including, without limitation, reversing, alleviating, suppressing the progress of, or preventing a disease or disorder to which the term applies, or one or more symptoms of the disease or disorder. In addition, “treatment” or “therapy” of inflammatory diseases in a mammal animal may include one or more of the following:
(1) arresting the development of inflammatory diseases;
(2) preventing the spread of inflammatory diseases;
(3) alleviating inflammatory diseases;
(4) preventing the recurrence of inflammatory diseases; and
(5) alleviating the symptoms of inflammatory diseases.

In the present invention, applicable inflammatory diseases may include, without limitation, inflammatory skin diseases and inflammatory diseases of tissues such as the stomach, esophagus, small intestine, urethra and conjunctiva. The inflammatory diseases may include, but are not limited to, diseases such as atopic dermatitis, psoriasis, urticaria, for example, chronic urticaria, hidradenitis suppurativa, acne, for example, acne conglobata, asthma, contact dermatitis, rhinitis, conjunctivitis, arthritis, bronchitis, bedsores, ulcers, cancer, ichthyosis, pemphigus, acne, lupus erythematosus, skin aging and skin wrinkles, and any disease that may be caused by an inflammatory response in the skin may be included in the range of the skin diseases of the present invention.

The biocompatible polypeptide may be, but is not limited to, a mussel or barnacle-derived adhesive protein, preferably a mussel-derived adhesive protein. In an embodiment, the mussels may be selected from the group consisting of, but is not limited to, Mytilus edulis, Mytilus galloprovincialis and Mytilus coruscus, and preferably may be Mytilus galloprovincialis.

In the present invention, the mussel adhesive protein is an adhesive protein derived from mussels, is an adhesive protein capable of self-adhesion in a humid or dry environment, and is preferably, but is not limited to, a recombinant mussel adhesive protein or a mutant of the mussel adhesive protein, and preferably, may include, without limitation, any mussel adhesive protein described in International Publication Nos. WO 2006/107183 A1 and WO 2005/092920. The mutant of the mussel adhesive protein may preferably be one which includes an additional sequence at the carboxyl terminal (C-terminal) or amino terminal (N-terminal) of the mussel adhesive protein, or one in which some amino acids are substituted with another amino acid, on the premise of maintaining the adhesion of the mussel adhesive protein. More preferably, it may be one in which a physiological functional peptide, for example, a polypeptide consisting of 3 to 25 amino acids including RGD is linked to the carboxyl terminal or amino terminal of the mussel adhesive protein, or one in which 1 to 100%, preferably 5 to 100%, more preferably 50 to 100% of the total number of tyrosine residues constituting the mussel adhesive protein is substituted with 3,4-dihydroxyphenyl-L-alanine (DOPA).

Examples of commercially available mussel adhesive proteins are, but are not limited to, BV_Serise, which is a mussel-derived recombinant antimicrobial adhesive protein marketed by Biovit Co., Ltd., located in Mapo-gu, Seoul, Korea, and a mussel adhesive protein sold by Kollodis BioSciences, Inc., located in North Augusta, South Carolina, USA.

In an embodiment, the mussel adhesive protein may be used as it is, or selected from the group consisting of FP-1, FP-2, FP-3, FP-4, FP-5, FP-6 and fragments thereof. In another embodiment, the mussel adhesive protein may be a fusion protein or fusion polypeptide in which two or more of FP-1, FP-2, FP-3, FP-4, FP-5, FP-6 and fragments thereof are fused to each other.

In a preferred embodiment, the mussel adhesive protein may used as it is, or may used as a fusion protein in which at least one second peptide selected from the group consisting of mussel adhesive proteins FP-1 (SEQ ID NOs: 1, 2, 3, 4 and 5), FP-2 (SEQ ID NO: 6) and FP-4 (SEQ ID NO: 11) and fragments of each protein is fused to the carbon terminal, the amine terminal or both terminals of a first peptide corresponding to FP-3 set forth in SEQ ID NO: 7, 8, 9 or 10, FP-5 set forth in SEQ ID NO: 12, 13, 14 or 15, or FP-6 set forth in SEQ ID NO: 16. The protein selected as the second peptide may further comprise a spacer at the carbon or amine terminal, and a preferred spacer may include an amino acid sequence set forth in any one of SEQ ID NO: 25 to SEQ ID NO: 28. As the spacer, a peptide in which SEQ ID NO: 25, 26, 27 or 28 is repeated a plurality of times, for example, two or more times, may be used.

Preferably, the first peptide is FP-5 comprising the amino acid sequence of SEQ ID NO: 12 to SEQ ID NO: 15, and the second peptide is FP-1 comprising the amino acid sequence of SEQ ID NO: 1 to SEQ ID NO: 5. More preferably, the mussel adhesive protein that may be used in the present invention is an adhesive protein in which a spacer of any one of SEQ ID NO: 25 to SEQ ID NO: 28 is conjugated to the amine or carbon terminal of FP-1 comprising the amino acid sequence of SEQ ID NO: 1 to SEQ ID NO: 5.

In another embodiment, the mussel adhesive protein may be, but is not limited to: FP-151 (SEQ ID NO: 17 to SEQ ID NO: 21) in which FP-1 is fused to both terminals of FP-5; FP-131 (SEQ ID NO: 22) in which FP-1 is fused to both terminals of FP-3; FP-251 (SEQ ID NO: 23) in which FP-2 and FP-1 are fused to both terminals of FP-5; or FP-353 (SEQ ID NO: 17 to SEQ ID NO: 24) in which FP-3 is fused to both terminals of FP-5.

In the present invention, the biocompatible polypeptide constituting the recombinant polypeptide, such as mussel adhesive protein, preferably has a form in which a functional peptide is added to the C-terminal or N-terminal of a mussel adhesive protein present in nature or a mussel adhesive protein designed recombinantly.

In an embodiment, the functional peptide may be derived from the group consisting of, but is not limited to, an antimicrobial peptide, an extracellular matrix protein and a growth factor, preferably an antimicrobial peptide.

In an embodiment, the biocompatible polypeptide, such as mussel adhesive protein, and the functional peptide may be conjugated directly or conjugated through a linker. The linker may be a peptide linker or a non-peptide linker.

When the linker is a peptide linker, it may include one or more amino acids, and may include, but is not limited to, for example, 1 to 10 amino acids, preferably 2 to 6 amino acids, for example, 2 amino acids. In a preferred embodiment, the linker may be a dipeptide of KL.

When the linker is a non-peptide linker, a biocompatible polymer in which two or more repeating units are conjugated may be used. The repeating units are linked to each other via any covalent bond rather than a peptide bond. The non-peptide linker may be selected from the group consisting of, but is not limited to, fatty acids, polysaccharides, high molecular weight polymers, low molecular weight compounds, nucleotides and combinations thereof.

In the present invention, as the functional peptide added to the biocompatible polypeptide constituting the recombinant polypeptide, any peptide derived from nature or artificially synthesized may be used without limitation. In an embodiment, the functional peptide may be derived from the group consisting of, but is not limited to, an antimicrobial peptide, an extracellular matrix protein and a growth factor, preferably an antimicrobial peptide. The antimicrobial peptide may exert an antimicrobial effect through a mechanism of destroying the cell membrane of microorganisms or permeating the cell membrane to inhibit metabolic functions, and any antimicrobial peptide that exerts an antimicrobial effect through a mechanism of destroying the cell membrane of microorganisms may all be used in the present invention. Preferably, the antimicrobial peptide to be conjugated to the adhesive protein may be optionally selected from antimicrobial peptides effective against Gram-positive bacteria as well as Gram-negative bacteria.

In an embodiment, the antimicrobial peptide may be selected from the group consisting of, but is not limited to, KLWKKWAKKWLKLWKA (SEQ ID NO: 29), FALALKALKKL (SEQ ID NO: 30), ILRWPWWPWRRK (SEQ ID NO: 31), AKRHHGYKRKFH (SEQ ID NO: 32), KWKLFKKIGAVLKVL (SEQ ID NO: 33), LVKLVAGIKKFLKWK (SEQ ID NO: 34), IWSILAPLGTTLVKLVAGIGQQKRK (SEQ ID NO: 35), GIGAVLKVLTTGLPALISWI (SEQ ID NO: 36), SWLSKTAKKGAVLKVL (SEQ ID NO: 37), KKLFKKILKYL (SEQ ID NO: 38), GLKKLISWIKRAAQQG (SEQ ID NO: 39), GWLKKIGKKIERVGQHTRDATIQGLGIAQQAANVAATAR (SEQ ID NO: 40), LKKLAKLALAF (SEQ ID NO: 41), KLLLKLLKKLLKLLKKK (SEQ ID NO: 42), THRPPMWSPVWP (SEQ ID NO: 43), GWLKKIGKWKIFKK (SEQ ID NO: 44), ILPWKWPWWPWRR (SEQ ID NO: 45), RRWWCRC (SEQ ID NO: 46), KLAKLAKKLAKLAK (SEQ ID NO: 47), LKKLAKLALAFILRWPWWPWRRK (SEQ ID NO: 48), RLLRRLLRRLLRRLLRRLLR (SEQ ID NO: 49), FLKLLKKLAAKLF (SEQ ID NO: 50), WLKKIGKKIERVGQHTRDATIQGLGIAQQAANVAATAR (SEQ ID NO: 51), and GTNNWWQSPSIQN (SEQ ID NO: 52). In another embodiment, the antimicrobial peptide may include, but is not limited to, magainin, which is an α-helical 23-amino acid peptide isolated from the skin of the African frog Xenopus laevis, dermaseptin, human defensin, cathelicidin LL-37, histatin, and the like.

In another embodiment, the extracellular matrix protein may be selected from the group consisting of, but is not limited to, collagen, fibronectin, laminin, and vitronectin, and the growth factor may be selected from the group consisting of, but is not limited to, epidermal growth factor (EGF), fibroblast growth factor (FGF), and vascular endothelial growth factor (VEGF).

In an embodiment, the functional peptide may be fused to the C-terminal, the N-terminal or both terminals of the biocompatible polypeptide, such as mussel adhesive protein.

In the present invention, in order to use a composition comprising a recombinant polypeptide in which a functional peptide is conjugated to a biocompatible polypeptide, such as mussel adhesive protein, as a therapeutic agent for inflammation, the composition may be used in the form of a lyophilized powder, a solution using lyophilized powder, a gel, or the like. An effective concentration for use as an anti-inflammatory agent may be easily determined by those skilled in the art through toxicity tests in each application field, such as oral, transdermal and intravenous applications.

Among the amino acids constituting the recombinant polypeptide disclosed in the present invention, tyrosine residues may be changed to DOPA and further to DOPA quinone through chemical modification. In an embodiment, the chemical modification of the recombinant polypeptide may be performed using, for example, a mushroom-derived tyrosinase enzyme capable of mediating such chemical modification.

In an embodiment, the biocompatible polypeptide, such as mussel adhesive protein, disclosed in the present invention may preferably be mass-produced by genetic engineering by inserting it into a conventional vector constructed for the purpose of expressing a foreign gene, but is not limited thereto. The vector may be appropriately selected or newly constructed depending on the type and characteristics of a host cell for producing the protein. A method of transforming the vector into a host cell and a method of producing a recombinant protein from a transformant may be easily performed by a conventional method. Methods such as selection and construction of the vector and transformation with the vector, and expression of recombinant proteins may be easily carried out by those skilled in the art to which the present invention belongs, and some modifications of conventional methods are also included in the present invention.

In an embodiment, the host cell may be a prokaryotic cell or a eukaryotic cell, and preferably may be, but is not limited to, an E. coli cell.

In the present invention, the term “gene” is used in the sense of including, without limitation, nucleic acid (for example, DNA) sequences containing coding sequences necessary to produce polypeptides, precursors or RNA (for example, rRNA and tRNA). In the present invention, the term “gene” includes both cDNA and genomic forms of genes. A genomic form or clone of a gene contains coding regions interrupted by non-coding sequences called “introns” or “insertion regions” or “insertion sequences.” A polypeptide may be encoded by a full-length coding sequence, or may be encoded by a portion of the coding sequence, so long as the full-length or fragment retains the desired active or functional properties (for example, enzymatic activity, ligand binding, signal introduction, immunogenicity, and the like). In addition, the term includes the coding region of a structural gene and sequences located adjacent to the coding region at the 5′ and 3′ ends at a distance of about 1 kb or more from the terminals such that the gene corresponds to the length of a full-length mRNA.

In the present invention, it is preferable to use an expression vector for efficient expression of a gene encoding the recombinant polypeptide of the present invention as the vector. In the present invention, “expression vector” refers to a recombinant vector capable of expressing a desired peptide in a suitable host cell, and refers to a genetic construct containing essential control elements operably linked to express a gene insert. The expression vector of the present invention may include expression control elements such as a promoter, an operator and a start codon as elements generally possessed by suitable expression vectors. The start codon and stop codon are generally considered to be part of the nucleotide sequence encoding a polypeptide, and must be functional in a subject when a genetic construct is introduced and must be in frame with a coding sequence. The promoter in the vector may be constitutive or inducible.

In addition, the vector may include a signal sequence for excretion of the fusion polypeptide in order to facilitate the isolation of the recombinant polypeptide of the present invention from a cell culture broth. Specific initiation signals may also be required for efficient translation of the inserted nucleic acid sequence. These signals include the start codon ATG and adjacent sequences. In some cases, exogenous translational control signals, which may include the start codon ATG, must be provided. These exogenous translation control signals and start codons may be of a variety of natural and synthetic sources. Expression efficiency may be increased by the introduction of suitable transcriptional or translational enhancers.

In an embodiment, all conventional expression vectors may be used without limitation as the vector. For example, plasmid DNA, phage DNA, and the like may be used. Specific examples of plasmid DNA include commercial plasmids such as pUC18 and pIDTSAMRT-AMP. Other examples of plasmids that may be used in the present invention are E. coli-derived plasmids (pET-22b(+), pYG601BR322, pBR325, pUC118 and pUC119), Bacillus subtilis-derived plasmids (pUB110 and pTP5), and yeast-derived plasmids (YEp13, YEp24 and YCp50). Specific examples of phage DNA are λ-phage (Charon4A, Charon21A, EMBL3, EMBL4, λgt10, λgt11 and λZAP). In addition, animal viruses such as retrovirus, adenovirus and vaccinia virus, and insect viruses such as baculovirus may be used. Since these expression vectors show different levels of protein expression and modification depending on a host cell, a host cell most suitable for the purpose may be selected and used.

In a preferred embodiment, the pET22b(+) vector may be used as the vector. The pET22b(+) vector is a vector for protein expression, wherein a recombinant MGFP-2 mutant is inserted into the Nde I/Hind III restriction enzyme site of the pET22b(+) vector, and an antimicrobial peptide is the Hind III/Xho I restriction enzyme site of the pET22b(+) vector. Since the pET22b(+) vector contains a T7 promoter, expression may be induced using isopropylthio-β-D-galactoside (IPTG), and an affinity ligand (for example, hexahisitidine) gene sequence for isolation and purification of proteins using affinity chromatography is present behind the Xho I restriction enzyme site.

In the present invention, a transformant may be obtained by introducing the vector into a suitable host cell. Types of hosts may include various bacteria such as the genus Esherichia, the genus Pseudomonas, the genus Ralstonia, the genus Alcaligenes, the genus Comamonas, the genus Burkholderia, the genus Agrobacterium, the genus Flabobacterium, the genus Vibrio, the genus Enterobacter, the genus Rhizobium, the genus Gluconobacter, the genus Acinetobacter, the genus Moraxella, the genus Nitrosomonas, the genus Aeromonas, the genus Paracoccus, the genus Bacillus, the genus Clostridium, the genus Lactobacillus, the genus Corynebacterium, the genus Arthrobacter, the genus Achromobacter, the genus Micrococcus, the genus Mycobacterium, the genus Streptococcus, the genus Streptococcus, the genus Streptomyces, the genus Actinomyces, the genus Nocardia and the genus Methylobacterium. In addition to the above bacteria, the hosts may include, but are not limited to, yeasts such as the genus Saccharomyces and the genus Candida, and various fungi.

For example, when a bacterium such as E. coli is used as the host, it is preferable that the recombinant vector itself is capable of autonomous replication in the host and has elements necessary for expression, such as a promoter, DNA containing a gene encoding the recombinant polypeptide, and a transcriptional stop sequence. Methods for introducing recombinant DNA into bacteria may include, but are not limited to, a calcium chloride method, an electroporation method, a spheroplast method, a lithium acetate method, and the like.

In an embodiment, the recombinant polypeptide included in the composition of the present invention may be prepared by a preparation method comprising the steps of: (a) constructing a vector comprising a gene encoding the recombinant polypeptide of the present invention; (b) transforming the vector into a host cell to prepare a transformant; and (c) culturing the transformant. In another embodiment, the preparation method may further comprise (d) isolating and purifying the recombinant polypeptide.

As a method for culturing the transformant, a conventional method used for culturing a host may be used. In addition, as the culture method, any method commonly used for culture of microorganisms, such as batch type, fluidized batch type, continuous culture and reactor type, may be used. The medium for the transformant obtained by using a bacterium such as E. coli as a host may include a complete medium or a synthetic medium, for example, LB medium, M9 medium, and the like. In addition, the recombinant polypeptide of the present invention may be accumulated in cells and recovered therefrom by culturing within the above-mentioned appropriate temperature range.

Carbon sources are necessary for the growth of microorganisms, and for example, sugars such as glucose, fructose, sucrose, maltose, galactose, and starch; lower alcohols such as ethanol, propanol and butanol; polyhydric alcohols such as glycerol; organic acids such as acetic acid, citric acid, succinic acid, tartaric acid, lactic acid and gluconic acid; fatty acids such as propionic acid, butanoic acid, pentanoic acid, hexanoic acid, heptanoic acid, octanoic acid, nonanoic acid, decanoic acid, undecanoic acid and dodecanoic acid may be used.

Nitrogen sources may include those derived from natural products such as peptone, meat juice, yeast extract, malt extract, casein hydrolysate and corn steep liquor, in addition to ammonium salts such as ammonia, ammonium chloride, ammonium sulfate and ammonium phosphate, for example. In addition, inorganic materials may include monopotassium phosphate, dipotassium phosphate, magnesium phosphate, magnesium sulfate, sodium chloride, and the like, for example. Antibiotics such as kanamycin, ampicillin, tetracycline, chloramphenicol and streptomycin may be added to the culture broth.

In addition, when a microorganism transformed using an expression vector having an inducible promoter is cultured, an inducer suitable for the type of promoter may be added to the culture medium. For example, the inducer may include isopropyl-β-D-thiogalactopyranoside (IPTG), tetracycline, indole acrylic acid (IAA), and the like. Acquisition and purification of the recombinant polypeptide of the present invention may be performed by, but are not limited to, centrifugation recovery of the cells or supernatant from the obtained culture, and alone or in an appropriate combination of cell disruption, extraction, affinity chromatography, cation or anion exchange chromatography, gel filtration, and the like.

In an embodiment, the transformant may be cultured in a conventional LB medium, and IPTG may be added to induce expression of the recombinant polypeptide. A preferred method for expressing a recombinant polypeptide may include, but is not limited to, culturing the transformant in LB (5 g/l yeast extract, 10 g/l tryptone and 10 g/l NaCl) medium, adding 0.5 mM to 4 mM IPTG thereto when the absorbance of the culture broth is 0.6 to 0.8 at 600 nm, and culturing it for 3 hours. The recombinant polypeptide expressed by the above method is produced in the form of an inclusion body, and the inclusion body may be solubilized to isolate and purify the recombinant polypeptide.

In a preferred embodiment, the composition of the present invention may be in the form of a pharmaceutical composition, wherein the pharmaceutical composition may comprise, without limitation, 0.001 to 10% by weight, 0.01 to 5% by weight, or 0.01 to 3% by weight, or 0.1 to 2% by weight, or 0.5 to 1.5% by weight of the recombinant polypeptide in which the functional peptide is conjugated to the biocompatible polypeptide, based on the total weight of the pharmaceutical composition.

The pharmaceutical composition of the present invention may further comprise other components that may give a synergistic effect to the effect of the recombinant polypeptide within a range that does not impair the effect of the present invention, in addition to the recombinant polypeptide. For example, it may comprise conventional adjuvants such as antioxidants, stabilizers, solubilizers, vitamins, pigments and flavorings, or carriers.

The route of administration of the pharmaceutical composition includes oral, intravenous, intramuscular, intraarterial, transdermal, subcutaneous, intraperitoneal, intranasal, intestinal, topical, sublingual or rectal methods, and for example, a topical application method by application may be applied. The parenteral methods include subcutaneous, intracutaneous, intravenous, intramuscular, intralesional injection or infusion techniques.

The pharmaceutical composition of the present invention may be administered in a pharmaceutically effective amount. In the present invention, the “pharmaceutically effective amount” refers to an amount sufficient to treat a disease at a reasonable benefit/risk ratio applicable to medical treatment, and the level of the effective dose may be determined depending on the factors including the individual type and severity, age, sex, and the type of disease, the activity of the drug, the sensitivity to the drug, the time of administration, the route of administration and the rate of the excretion, the duration of treatment, and the drugs used simultaneously, and other factors well known in the medical field. The composition of the present invention may be administered as an individual therapeutic agent or in combination with other therapeutic agents, and may be administered sequentially or concurrently with conventional therapeutic agents. In addition, it may be administered single or multiple. Considering all of the above factors, it is important to administer an amount that can achieve the maximum effect in a minimal amount without side effects, which may be easily determined by those skilled in the art.

The pharmaceutical composition of the present invention may be applied without limitation to tissues, organs or individuals for the purpose of anti-inflammatory action, and may be applied to anything. For example, the pharmaceutical composition of the present invention may be used not only for humans but also for non-human animals such as monkeys, dogs, cats, rabbits, guinea pigs, rats, mice, cattle, sheep, pigs and goats, birds, fish, and the like.

The pharmaceutical composition of the present invention may comprise one or more active ingredients exhibiting the same or similar functions in addition to the recombinant polypeptide. The pharmaceutical composition may be formulated and used in the form of oral formulations such as powders, granules, tablets, capsules, suspensions, emulsions and syrups, external preparations, suppositories and sterile injectable solutions according to conventional methods, respectively.

Formulations for parenteral administration may be formulated and used in the form of powders, granules, tablets, capsules, sterilized aqueous solutions, liquids and solutions, non-aqueous solutions, suspensions, emulsions, syrups, external preparations such as suppositories and aerosols, and sterile injection preparations according to conventional methods, respectively, and preferably, a pharmaceutical composition for external application to the skin such as creams, gels, patches, sprays, ointments, plasters, lotions, liniments, pastes, or cataplasmas may be prepared and used, but is not limited thereto. As the non-aqueous solutions and suspensions, propylene glycol, polyethylene glycol, vegetable oils such as olive oil, injectable esters such as ethyl oleate, and so on may be used. As a base for suppositories, witepsol, macrogol, Tween 61, cacao butter, laurin butter, glycerogelatin, and so on may be used.

The pharmaceutical composition of the present invention may further comprise adjuvants such as preservatives, stabilizers, hydrating agents or emulsifying accelerators and salts and/or buffers for controlling osmotic pressure, and other therapeutically useful substances, and may be formulated according to mixing, granulating or coating methods.

The dosage of the pharmaceutical composition of the present invention may vary depending on various factors including the individual age, body weight, general health and sex, the time of administration, the route of administration, the rate of the excretion, the combination of drugs, and the severity of a specific disease.

In addition, the pharmaceutical composition of the present invention may be used alone or in combination with methods using surgery, radiotherapy, hormone therapy, chemotherapy, and biological response modifiers. In addition, the pharmaceutical composition may be administered at a dosage within the range of 1 to 20,000 mg/kg, such as 1 to 10,000 mg/kg, 1 to 1,000 mg/kg, or 1 to 200 mg/kg for adults, and if the composition is an external preparation, it is recommended to apply the composition 1 to 5 times a day in an amount of 1.0 to 3.0 ml for adults and continue for 1 month or more, but the dosage is not intended to limit the scope of the present invention. The pharmaceutical composition may be used alone or in combination with methods using surgery, radiotherapy, hormone therapy, chemotherapy, and biological response modifiers.

In another preferred embodiment, the composition of the present invention may be in the form of a cosmetic composition.

The cosmetic composition of the present invention may be prepared in liquid or solid form using bases, adjuvants and additives commonly used in the field of cosmetics. Cosmetics in liquid or solid form may include, but are not limited to, forms such as toners, creams and lotions, for example.

The cosmetic composition of the present invention may comprise commonly used components, and for example, may comprise conventional adjuvants such as antioxidants, preservatives, stabilizers, solubilizers, vitamins, pigments and flavorings, and carriers.

In addition, the cosmetic composition of the present invention may further comprise, but is not limited to, wrinkle-improving cosmetics, whitening cosmetics, sunscreens, and light scattering agents in order to add or enhance functionality.

The cosmetic composition of the present invention may also be prepared in any formulations conventionally prepared in the art, and may be formulated into, but is not limited to, for example, a solution, a suspension, an emulsion, a paste, a gel, a cream, a lotion, a powder, a soap, a surfactant-containing cleansing, an oil, a powder foundation, an emulsion foundation, a wax foundation, a spray, and the like. More specifically, it may be prepared in a formulation of a skin softener, a nutritional toner, a nutrition cream, a massage cream, an essence, an eye cream, a cleansing cream, a cleansing foam, a cleansing water, a pack, a spray or a powder.

When the formulation of the present invention is a paste, a cream, or a gel, animal oil, vegetable oil, wax, paraffin, starch, tragacanth, cellulose derivative, polyethylene glycol, silicone, bentonite, silica, talc, or zinc oxide, and the like may be used as the carrier ingredient.

If the formulation of the present invention is a powder or a spray, a lactose, a talc, a silica, an aluminum hydroxide, a calcium silicate, or a polyamide powder may be used as the carrier ingredient, in particular in the case of a spray, a propellant such as chlorofluorohydrocarbon, propane/butane, or dimethyl ether may be further included.

If the formulation of the present invention is a solution or an emulsion, a solvent, a solubilizer, or an emulsifier is used as the carrier ingredient, such as water, ethanol, isopropanol, ethyl carbonate, ethyl acetate, benzyl alcohol, benzyl benzoate, propylene glycol, 1,3-butylene glycol oil, glycerol aliphatic ester, polyethylene glycol, or fatty acid esters of sorbitan.

If the formulation of the present invention is a suspension, a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, tragacanth, or the like may be used.

When the formulation of the present invention is a surfactant-containing cleansing, aliphatic alcohol sulfate, aliphatic alcohol ether sulfate, sulfosuccinic acid monoester, imidazolinium derivative, methyltaurate, sarcosinate, fatty acid amide ether sulfate, alkylamidobetaine, aliphatic alcohol, fatty acid glyceride, fatty acid diethanolamide, vegetable oil, lanolin derivative, or ethoxylated glycerol fatty acid ester, and the like may be used as the carrier ingredient.

The cosmetic composition of the present invention may be used alone or overlapping, or may be used overlapping with other cosmetic compositions other than the present invention. In addition, the cosmetic composition for cell regeneration or anti-inflammation according to the present invention may be used according to a conventional method of use, and the number of times of use may be varied according to the user's skin condition or preference.

In another preferred embodiment, the present invention provides a method of suppressing inflammation, comprising the step of: administering to a subject the recombinant polypeptide in which a functional peptide is conjugated to a biocompatible polypeptide, or a composition, for example, a pharmaceutical composition or a cosmetic composition, comprising the recombinant polypeptide.

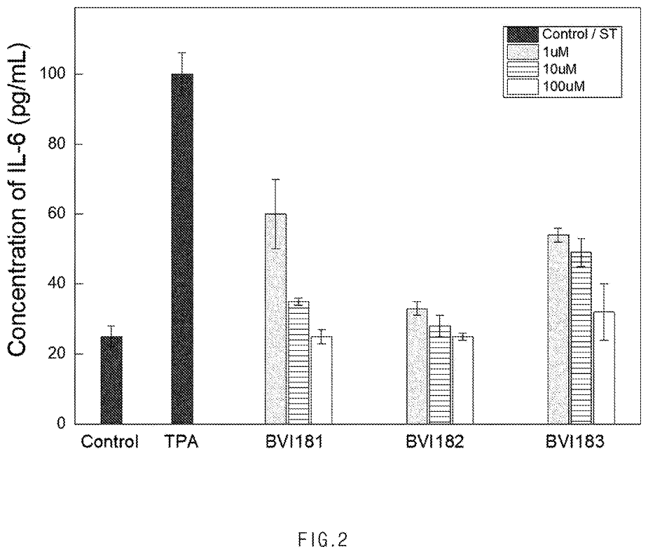

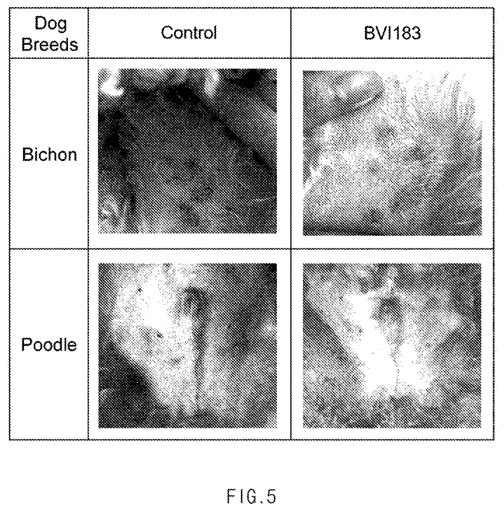

In an embodiment of the present invention, the present inventors confirmed that the functional protein composition effectively inhibits inflammatory cytokines that induce inflammation-related diseases. In a preferred embodiment, it was confirmed that the composition of the present invention has excellent anti-inflammatory activity by remarkably inhibiting interleukin-6 (IL-6), which is an inflammatory cytokine expressed by 12-O-tetradecanoylphorbol-13-acetate (TPA) that induces skin inflammation, and TNF-α, which is a tumor necrosis factor, and reducing ear thickness in TPA-treated mice similarly to that of normal control groups (see FIGS. 2 to 4). IL-6 is a cytokine that functions in both innate and adaptive immunity and is produced in B cells, T cells, fibroblasts, vascular endothelial cells, and skin keratinocytes, and since it is involved in various immune and inflammatory responses, the decrease in IL-6 expression level has a very important meaning. In another embodiment, the present inventors confirmed that when the functional protein composition was applied to dogs with inflammatory diseases, inflammation was remarkably reduced (see FIG. 5).

The association of TNF-α and IL-6 with various inflammatory diseases is known in the art. TNF-α is a type of cytokine that is involved in the inflammatory response and is a member of the acute phase response, and plays a role in regulating immune cells. TNF-α induces apoptosis, induces sepsis through the production of IL-1 and IL-6, and causes various human inflammatory diseases due to its abnormal regulation. In addition, IL-6 is a type of cytokine produced in an early stage of an inflammatory response and is a physiologically active protein released by cells to give signals to other cells. The actions of IL-6 are mainly related to the immune response, but also promotes several inflammatory responses due to infection, trauma, burns or tissue damage leading to inflammation.

An increase in TNF-α has been found in patients with atopic dermatitis and correlates with the severity of dermatitis. In addition, it was confirmed that since a significant correlation was found between the concentration of TNF-α and histamine that induces itching, excessive secretion of TNF-α may affect the pathophysiological mechanism of atopic dermatitis (Sumimoto, S., Kawai, M., Kasajima, Y., & Hamamoto, T. (1992) Archives of Disease in Childhood, 67 (3), 277-279. doi: 10.1136/adc.67.3.277). In addition, serum IgE is increased in 80% or more of patients with atopic dermatitis, and this numerical value is proportional to the clinical severity of atopic dermatitis, and IL-6 is known to amplify IgE production.

In addition, IL-6 has long been studied as being related to the pathogenesis of psoriasis, and increased levels of TNF-α and IL-6 are known to be characteristic of psoriasis (A. Castells-Rodellas, J. V. et al., Acta Dermato-Venereologica, vol. 72, no. 3, pp. 165-168, 1992; P. Neuner, et al., Journal of Investigative Dermatology, vol. 97, no. 1, pp. 27-33, 1991; R. M. Grossman, et al., Proceedings of the National Academy of Sciences of the United States of America, vol. 86, no. 16, pp. 6367-6371, 1989). In psoriasis, IL-6 is known to activate the STAT3 pathway to affect the expression of genes controlling the survival and proliferation of keratinocytes. Infliximab, which reduces the concentration of TNF-α, has been developed for the treatment of psoriasis, and IL-6 inhibitors had a successful therapeutic effect in patients with psoriasis, especially patients with pustular psoriasis (Andrea Saggini, et al., Journal of Immunology Research, vol. 2014, Article ID 964069, 10 pages, 2014. https://doi.org/10.1155/2014/964069).

In addition, it is known that the concentration of IL-6 is significantly increased in patients with chronic urticaria (Kasperska-Zajac, et al., Clinical & Experimental Allergy, 41 (10), 1386-1391, 2011, 111/j.1365-2222.2011.03789.x)

In addition, it is known that the level of IL-6 is increased in the skin of patients with hidradenitis suppurativa, which is an autoinflammatory disease (Haoxiang Xu et al., Postepy Dermatol Alergol. 2017 February; 34 (1): 82-84), and in particular, it has been shown that administration of infliximab, which reduces the concentration of TNF-α, to patients with severe hidradenitis suppurativa may be an effective therapeutic alternative (J. M. Fernandez-Vozmediano et al., Dermatology, 215 (1), 41-44, 2007, doi: 10.1159/000102032).

In addition, acne is an inflammatory condition caused by an increase in specific cytokines such as TNF and IL-1B, and Propionibacterium acne is known to induce inflammation by stimulating TNF production from keratinocytes. Therefore, it is contemplated that the use of TNF inhibitors may have a therapeutic role (Freja Laerke Sand et al., JAMA Dermatol. 2013; 149 (11): 1306-1307. doi: 10.1001/jamadermatol.2013.6678).

Hereinafter, the present invention will be described in more detail by way of examples.

However, the following examples are provided to explain preferred embodiments of the present invention, and the scope of the present invention is not limited by the following specific embodiments provided only for illustrative purposes. As disclosed in the present invention, it is apparent that functionally equivalent products, compositions and methods are included within the scope of the present invention.

### Example 1: Cloning of Genes Encoding Recombinant Polypeptides

In order to prepare compositions comprising the recombinant polypeptides of the present invention, three gene sequences in which a functional peptide sequence was added to the C-terminal or N-terminal site of the mussel adhesive protein were designed and template DNAs were synthesized, and then PCR was performed, and these were inserted into the final pET22b(+) vectors. Each of the three recombinant polypeptides has a structure in which the mussel adhesive protein of SEQ ID NO: 20, the spacer of SEQ ID NO: 25, and the polypeptide of any one of SEQ ID NOs: 29, 31 and 41 are fused. A specific form of the vector is a form of a fusion polypeptide in which a mussel adhesive protein/functional peptide is inserted into a pET22b(+) vector. Since the pET22b(+) vector contains a T7 promoter, expression may be induced using isopropylthio-β-D-galactoside (IPTG). The three recombinant polypeptides were named BVI181, BVI182 and BVI183, respectively.

### Example 2: Preparation and Culture of Transformants Producing Recombinant Polypeptides

Competent cells were prepared using CaCl₂) buffer in E. coli DH5a cells for cloning and E. coli BL21 (DE3) cells used for protein expression, respectively, and then transformed with the pET22b(+) mussel adhesive protein/functional peptide vector constructed in Example 1 by the heat shock (left at 42° C. for 1 minute) method. Selection of transformed colonies was performed using ampicillin (Goldbio), and from this, transformants producing E. coli Dh5α pET22b(+) mussel adhesive protein/functional peptide and E. coli BL21 (DE3) mussel adhesive protein/functional peptide were obtained.

### Example 3: Production and Obtaining of Recombinant Polypeptides

In order to produce the recombinant polypeptides BVI181, BVI182 and BVI183, E. coli BL21/pET22b(+) transformants were cultured in LB medium (5 g/l yeast extract, 10 g/l tryptone, and 10 g/l NaCl), and expression of the recombinant polypeptides was induced by adding IPTG thereto when an appropriate O.D. value was reached. Each of the recombinant polypeptides BVI181, BVI182 and BVI183 was expressed, and then isolated and purified to obtain final high-purity proteins.

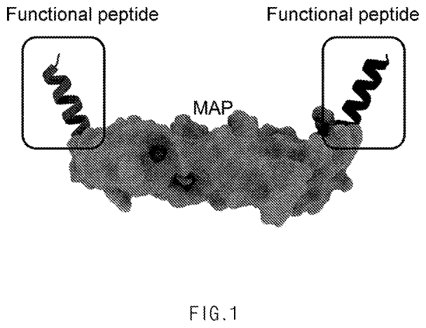

The amino acid sequences of the recombinant polypeptides BVI181, BVI182 and BVI183 were analyzed with an amino acid analyzer (Hewlett Packard, USA) to confirm that they were the same protein, and the structure of the polypeptide BVI183 among the three recombinant polypeptides was predicted through the peptide data bank (PDB) (FIG. 1).

### Example 4: IL-6 and TNF-α Secretion Inhibitory Effect of the Compositions of the Present Invention

IL-6 is a cytokine involved in immune and inflammatory responses, and it was confirmed whether the recombinant polypeptides BVI181, BVI182 and BVI183 of the present invention could inhibit IL-6 and TNF-α. To this end, 100 μl of a solution obtained by diluting the capture antibody for mouse IL-6 and TNF-α in a coating buffer was dispensed into a tissue culture dish (Costar; Cambridge, MA, USA), and left at 4° C. for about 12 to 20 hours, and then washed with a washing buffer. 250 μl of assay diluent (PBS-T; 0.01 M-PBA, Sigma-aldrich+0.05% Tween 20, Sigma-aldrich) was dispensed into the prepared sample, washed, and prepared. 100 μl of the detection antibody was dispensed into the prepared tissue culture dish and washed, and then 100 μl of avidin-horseradish peroxidase solution was dispensed thereinto, and then substrate solution containing tetramethylbenzidine was added thereto and reacted. Thereafter, the reaction was stopped by adding 50 μl of a stop solution (2% sulfuric acid, Sigma-aldrich). Optical density was measured at 450 nm using a microplate reader (Model Multiskan Sky Microplate Spectrophotometer; ThermoFisher).

As a result of applying compositions comprising the recombinant polypeptides BVI181, BVI182 and BVI183 of the present invention at concentrations of 1 μM, 10 μM and 100 μM, respectively, it was confirmed that IL-6 was all inhibited from a low concentration of 1 μM in the samples to which the compositions of the present invention were applied, compared to the inflammatory model (100 μg/ml), and in particular, it was confirmed that the inhibition results of each of the recombinant polypeptides BVI181, BVI182 and BVI183 was 25±2, 25±1 and 32±8 μg/ml at 100 μM, and thus IL-6 was remarkably reduced (FIG. 2).

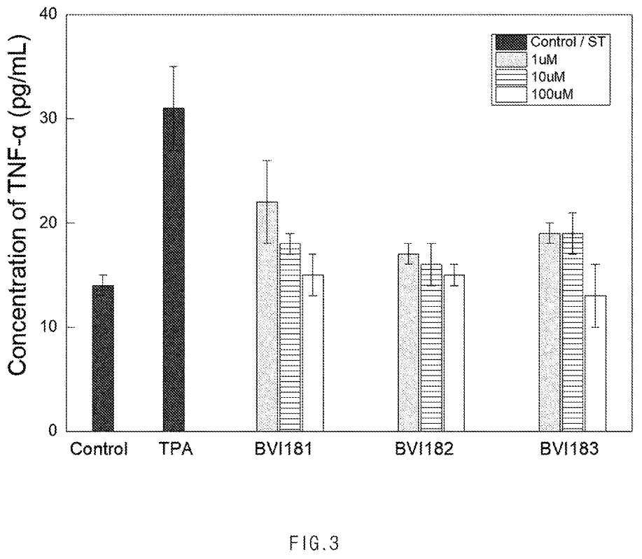

In addition, as a result of applying compositions comprising the recombinant polypeptides BVI181, BVI182 and BVI183 of the present invention at concentrations of 1 μM, 10 μM and 100 μM, it was confirmed that TNF-α was all inhibited from a low concentration of 1 μM in the samples to which the compositions of the present invention were applied, compared to the inflammatory model (30 pg/ml), and in particular, it was confirmed that the inhibition results of each of the recombinant polypeptides BVI181, BVI182 and BVI183 was 15±2, 15±1 and 13±3 μg/ml at 100 μM, and thus TNF-α was remarkably reduced (FIG. 3).

### Example 5: Inflammation-Alleviating Effect of the Composition of the Present Invention in Mice with TPA-Induced Ear Edema

The TPA-induced ear edema mouse model is a model capable of observing erythema and edema due to acute inflammation, and an inflammation-alleviating effect was observed after treating this model with a composition comprising each of the recombinant polypeptides BVI181, BVI182 and BVI183. To this end, the ears of BALB/c mice were first treated with TPA, and then contacted with a composition comprising each of the recombinant polypeptides BVI181, BVI182 and BVI183 at a concentration of 10 μM for 1 hour a day for 3 days, and observed for the inflammation-alleviating effect.

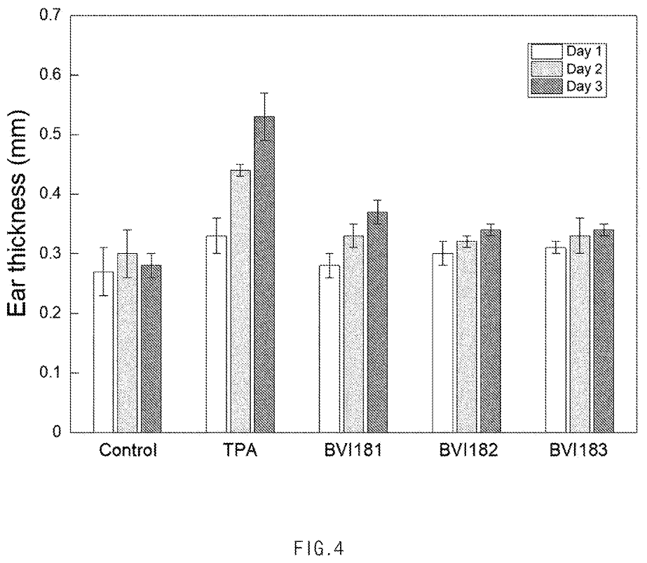

As a result, the ear thickness was all similar to that of normal mice from day 1 after treatment with each of the BVI181, BVI182 and BVI183 compositions, and from this, the inflammation-alleviating effect of the compositions of the present invention was confirmed. Specifically, it was confirmed that on day 3 after treatment with each of the BVI181, BVI182 and BVI183 compositions, the ear thickness was relieved from 0.53±0.02 mm in the group not treated with the compositions to 0.37±0.04, 0.34±0.01 and 0.34±0.01 mm, respectively, and was similar to the normal mouse ear thickness of 0.28±0.02 mm (FIG. 4).

### Example 6: Dermatitis-Alleviating Effect of a Composition Comprising the Recombinant Polypeptide BVI183 in Dogs

Dogs suffering from bacterial dermatitis and allergic dermatitis were treated with the composition comprising the recombinant polypeptide BVI183, respectively, and it was observed whether there was an anti-inflammatory effect. Among the dogs used in the experiment, the bichon suffers from allergic dermatitis, and shows itching as a typical symptom, which is known to be caused mainly by protein intake or dust mite secretions. In addition, the poodle suffers from bacterial dermatitis such as pyoderma or skin bacterial infections, which is known to cause symptoms such as pruritus and keratoderma due to bacterial overgrowth on the skin.

FIG. 5 shows the appearance observed 4 days after applying a composition comprising the recombinant polypeptide BVI183 to the skin of dogs (breeds: bichon and poodle) with dermatitis once a day in the form of a spray. It can be seen that dermatitis is rapidly reduced after application of the BVI183 composition, and in particular, it was observed that allergic dermatitis was alleviated and hair grew back in the bichon.

This research was conducted with the support of the Innovation Field Startup Package [B0080827000433] of the Ministry of SMEs and Startups of the Republic of Korea.

1. A pharmaceutical composition for preventing or treating inflammatory diseases, comprising a recombinant polypeptide in which a functional peptide is conjugated to a biocompatible polypeptide.

2. The pharmaceutical composition according to claim 1, wherein the inflammatory diseases are selected from the group consisting of inflammatory skin diseases and inflammatory diseases of the stomach, esophagus, small intestine, urethra and conjunctiva.

3. The pharmaceutical composition according to claim 1, wherein the inflammatory diseases are selected from the group consisting of atopic dermatitis, psoriasis, asthma, contact dermatitis, rhinitis, conjunctivitis, arthritis, bronchitis, bedsores, ulcers, cancer, ichthyosis, pemphigus, acne, lupus erythematosus, skin aging, and skin wrinkles.

4. The pharmaceutical composition according to claim 1, wherein the biocompatible polypeptide is a mussel adhesive protein or a barnacle adhesive protein.

5. The pharmaceutical composition according to claim 4, wherein the mussel is selected from the group consisting of Mytilus edulis, Mytilus galloprovincialis, and Mytilus coruscus.

6. The pharmaceutical composition according to claim 4, wherein the mussel adhesive protein is a protein in which a second peptide selected from the group consisting of FP-1 comprising the sequence of any one of SEQ ID NO: 1 to SEQ ID NO: 5, FP-2 comprising the sequence of SEQ ID NO: 6 and FP-4 comprising the sequence of SEQ ID NO: 11 is fused to the carbon terminal, the amine terminal or both terminals of a first peptide selected from the group consisting of FP-3 comprising the sequence of any one of SEQ ID NO: 7 to SEQ ID NO: 10, FP-5 comprising the sequence of any one of SEQ ID NO: 12 to SEQ ID NO: 15 and FP-6 comprising the sequence of SEQ ID NO: 16.

7. The pharmaceutical composition according to claim 4, wherein the mussel adhesive protein is selected from the group consisting of: FP-151 (SEQ ID NO: 17 to SEQ ID NO: 21) in which FP-1 is fused to both terminals of FP-5; FP-131 (SEQ ID NO: 22) in which FP-1 is fused to both terminals of FP-3; FP-251 (SEQ ID NO: 23) in which FP-2 and FP-1 are fused to both terminals of FP-5; and FP-353 (SEQ ID NO: 17 to SEQ ID NO: 24) in which FP-3 is fused to both terminals of FP-5.

8. The pharmaceutical composition according to claim 1, wherein the functional peptide is selected from the group consisting of an antimicrobial peptide, an extracellular matrix protein, and a growth factor.

9. The pharmaceutical composition according to claim 8, wherein the functional peptide is an antimicrobial peptide.

10. The pharmaceutical composition according to claim 1, wherein the biocompatible polypeptide and the functional peptide are conjugated directly or are conjugated through a peptide or non-peptide linker.

11. The pharmaceutical composition according to claim 9, wherein the antimicrobial peptide is selected from the group consisting of KLWKKWAKKWLKLWKA (SEQ ID NO: 29), FALALKALKKL (SEQ ID NO: 30), ILRWPWWPWRRK (SEQ ID NO: 31), AKRHHGYKRKFH (SEQ ID NO: 32), KWKLFKKIGAVLKVL (SEQ ID NO: 33), LVKLVAGIKKFLKWK (SEQ ID NO: 34), IWSILAPLGTTLVKLVAGIGQQKRK (SEQ ID NO: 35), GIGAVLKVLTTGLPALISWI (SEQ ID NO: 36), SWLSKTAKKGAVLKVL (SEQ ID NO: 37), KKLFKKILKYL (SEQ ID NO: 38), GLKKLISWIKRAAQQG (SEQ ID NO: 39), GWLKKIGKKIERVGQHTRDATIQGLGIAQQAANVAATAR (SEQ ID NO: 40), LKKLAKLALAF (SEQ ID NO: 41), KLLLKLLKKLLKLLKKK (SEQ ID NO: 42), THRPPMWSPVWP (SEQ ID NO: 43), GWLKKIGKWKIFKK (SEQ ID NO: 44), ILPWKWPWWPWRR (SEQ ID NO: 45), RRWWCRC (SEQ ID NO: 46), KLAKLAKKLAKLAK (SEQ ID NO: 47), LKKLAKLALAFILRWPWWPWRRK (SEQ ID NO: 48), RLLRRLLRRLLRRLLRRLLR (SEQ ID NO: 49), FLKLLKKLAAKLF (SEQ ID NO: 50), WLKKIGKKIERVGQHTRDATIQGLGIAQQAANVAATAR (SEQ ID NO: 51), and GTNNWWQSPSIQN (SEQ ID NO: 52).

12. A cosmetic composition for ameliorating inflammatory diseases, comprising a recombinant polypeptide in which a functional peptide is conjugated to a biocompatible polypeptide.

13. The cosmetic composition according to claim 12, wherein the biocompatible polypeptide is a mussel adhesive protein.

14. The cosmetic composition according to claim 12, wherein the functional peptide is an antimicrobial peptide.

15. The cosmetic composition according to claim 12, wherein the inflammatory diseases are selected from the group consisting of atopic dermatitis, psoriasis, urticaria, hidradenitis suppurativa, acne, asthma, contact dermatitis, rhinitis, conjunctivitis, arthritis, bronchitis, bedsores, ulcers, cancer, ichthyosis, pemphigus, acne, lupus erythematosus, skin aging, and skin wrinkles.


SEQUENCE LISTING (52 sequences)

SEQ ID NO: 1 - protein, 10 aa, Artificial Sequence
  Foot Protein type 1 (FP-1, Mytilus edulis)
AKPSYPPTYK

SEQ ID NO: 2 - protein, 20 aa, Artificial Sequence
  2 times repeated sequence derived from Foot Protein type 1 (FP-1)
AKPSYPPTYKAKPSYPPTYK

SEQ ID NO: 3 - protein, 30 aa, Artificial Sequence
  3 times repeated sequence derived from Foot Protein type 1 (FP-1)
AKPSYPPTYKAKPSYPPTYKAKPSYPPTYK

SEQ ID NO: 4 - protein, 40 aa, Artificial Sequence
  4 times repeated sequence derived from Foot Protein type 1 (FP-1)
AKPSYPPTYKAKPSYPPTYKAKPSYPPTYKAKPSYPPTYK

SEQ ID NO: 5 - protein, 60 aa, Artificial Sequence
  6 times repeated sequence derived from Foot Protein type 1 (FP-1)
AKPSYPPTYKAKPSYPPTYKAKPSYPPTYKAKPSYPPTYKAKPSYPPTYKAKPSYPPTYK

SEQ ID NO: 6 - protein, 39 aa, Artificial Sequence
  partial sequence of Foot Protein type 2 (FP-2, Mytilus californianus)
EVHACKPNPCKNNGRCYPDGKTGYKCKCVGGYSGPTCAC

SEQ ID NO: 7 - protein, 52 aa, Artificial Sequence
  Foot Protein type 3 (FP-3, Mytilus edulis)
ADYYGPKYGPPRRYGGGNYNRYGGSRRYGGYKGWNNGWKRGRWGRKYYEFEF

SEQ ID NO: 8 - protein, 46 aa, Artificial Sequence
  Foot Protein type 3 (FP-3, Mytilus galloprovincialis mgfp-3A)
ADYYGPKYGPPRRYGGGNYNRYGRRYGGYKGWNNGWKRGRWGRKYY

SEQ ID NO: 9 - protein, 50 aa, Artificial Sequence
  Foot Protein type 3 (FP-3, Mytilus edulis mefp-3F)
ADYYGPNYGPPRRYGGGNYNRYNGYGGGRRYGGYKGWNNGWNRGRRGKYW

SEQ ID NO: 10 - protein, 44 aa, Artificial Sequence
  Foot Protein type 3 (FP-3, Mytilus californianus)
GAYKGPNYNYPWRYGGKYNGYKGYPRGYGWNKGWNKGRWGRKYY

SEQ ID NO: 11 - protein, 60 aa, Artificial Sequence
  partial sequence from Foot Protein type 4 (Mytilus californianus)
GHVHRHRVLHKHVHNHRVLHKHLHKHQVLHGHVHRHQVLHKHVHNHRVLHKHLHKHQVLH

SEQ ID NO: 12 - protein, 75 aa, Artificial Sequence
  Foot Protein type 5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 13 - protein, 76 aa, Artificial Sequence
  Foot Protein 5 (FP-5, Mytilus edulis)
SSEEYKGGYYPGNTYHYHSGGSYHGSGYHGGYKGKYYGKAKKYYYKYKNSGKYKYLKKARKYHRKGYKKYYGGGSS

SEQ ID NO: 14 - protein, 71 aa, Artificial Sequence
  Foot Protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 15 - protein, 76 aa, Artificial Sequence; identical to SEQ ID NO: 13
  mussel adhesive Foot Protein type 5 (FP-5, Mytilus galloprovincialis)

SEQ ID NO: 16 - protein, 99 aa, Artificial Sequence
  mussel adhesive Foot Protein type 6 (FP-6)
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 17 - protein, 120 aa, Artificial Sequence
  hybrid mussel adhesive protein (FP-151, MEFP-5 based)
AKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKGGGGGG

SEQ ID NO: 18 - protein, 166 aa, Artificial Sequence
  hybrid mussel adhesive protein (FP-151, MEFP-5 based)
AKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKSGSGSGSGSGSG

SEQ ID NO: 19 - protein, 194 aa, Artificial Sequence
  hybrid mussel adhesive protein (FP-151, MEFP-5 based)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 20 - protein, 196 aa, Artificial Sequence
  hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 21 - protein, 192 aa, Artificial Sequence
  hybrid mussel adhesive protein (FP-151, MCFP-5 based)
AKPSYPPTYKAKPSYPPTYKAKPSYPPTYKAKPSYPPTYKAKPSYPPTYKAKPSYPPTYKYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 22 - protein, 177 aa, Artificial Sequence
  hybrid mussel adhesive protein (FP-131)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKL

SEQ ID NO: 23 - protein, 180 aa, Artificial Sequence
  hybrid mussel adhesive protein (FP-251)
MEVHACKPNPCKNNGRCYPDGKTGYKCKCVGGYSGPTCACSSEEYKGGYYPGNSNHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKK

SEQ ID NO: 24 - protein, 182 aa, Artificial Sequence
  hybrid mussel adhesive protein (FP-353)
GCRADYYGPKYGPPRRYGGGNYNRYGGSRRYGGYKGWNNGWKRGRWGRKYYEFEFYDGYSDGYYPGSAYNYPSGSHGYHGHGYKGKYYGKGKKYYYKYKRTGKYKYLKKARKYHRKGYKKYYGGGSSGCRADYYGPKYGPPRRYGGGNYNRYGGSRRYGGYKGWNNGWKRGRWGRKYYEFEF

SEQ ID NO: 25 - protein, 2 aa, Artificial Sequence
  spacer group
KL

SEQ ID NO: 26 - protein, 4 aa, Artificial Sequence
  spacer group
KLKL

SEQ ID NO: 27 - protein, 6 aa, Artificial Sequence
  spacer group
SGSGSG

SEQ ID NO: 28 - protein, 12 aa, Artificial Sequence
  spacer group
SGSGSGSGSGSG

SEQ ID NO: 29 - protein, 16 aa, Artificial Sequence
  anti-microbial peptide
KLWKKWAKKWLKLWKA

SEQ ID NO: 30 - protein, 11 aa, Artificial Sequence
  anti-microbial peptide
FALALKALKKL

SEQ ID NO: 31 - protein, 12 aa, Artificial Sequence
  anti-microbial peptide
ILRWPWWPWRRK

SEQ ID NO: 32 - protein, 12 aa, Artificial Sequence
  anti-microbial peptide
AKRHHGYKRKFH

SEQ ID NO: 33 - protein, 15 aa, Artificial Sequence
  anti-microbial peptide
KWKLFKKIGAVLKVL

SEQ ID NO: 34 - protein, 15 aa, Artificial Sequence
  anti-microbial peptide
LVKLVAGIKKFLKWK

SEQ ID NO: 35 - protein, 25 aa, Artificial Sequence
  anti-microbial peptide
IWSILAPLGTTLVKLVAGIGQQKRK

SEQ ID NO: 36 - protein, 20 aa, Artificial Sequence
  anti-microbial peptide
GIGAVLKVLTTGLPALISWI

SEQ ID NO: 37 - protein, 16 aa, Artificial Sequence
  anti-microbial peptide
SWLSKTAKKGAVLKVL

SEQ ID NO: 38 - protein, 11 aa, Artificial Sequence
  anti-microbial peptide
KKLFKKILKYL

SEQ ID NO: 39 - protein, 16 aa, Artificial Sequence
  anti-microbial peptide
GLKKLISWIKRAAQQG

SEQ ID NO: 40 - protein, 39 aa, Artificial Sequence
  anti-microbial peptide
GWLKKIGKKIERVGQHTRDATIQGLGIAQQAANVAATAR

SEQ ID NO: 41 - protein, 11 aa, Artificial Sequence
  anti-microbial peptide
LKKLAKLALAF

SEQ ID NO: 42 - protein, 17 aa, Artificial Sequence
  anti-microbial peptide
KLLLKLLKKLLKLLKKK

SEQ ID NO: 43 - protein, 12 aa, Artificial Sequence
  anti-microbial peptide
THRPPMWSPVWP

SEQ ID NO: 44 - protein, 14 aa, Artificial Sequence
  anti-microbial peptide
GWLKKIGKWKIFKK

SEQ ID NO: 45 - protein, 13 aa, Artificial Sequence
  anti-microbial peptide
ILPWKWPWWPWRR

SEQ ID NO: 46 - protein, 7 aa, Artificial Sequence
  anti-viral peptide
RRWWCRC

SEQ ID NO: 47 - protein, 14 aa, Artificial Sequence
  anti-microbial peptide
KLAKLAKKLAKLAK

SEQ ID NO: 48 - protein, 23 aa, Artificial Sequence
  anti-microbial peptide
LKKLAKLALAFILRWPWWPWRRK

SEQ ID NO: 49 - protein, 20 aa, Artificial Sequence
  anti-microbial peptide
RLLRRLLRRLLRRLLRRLLR

SEQ ID NO: 50 - protein, 13 aa, Artificial Sequence
  anti-microbial peptide
FLKLLKKLAAKLF

SEQ ID NO: 51 - protein, 38 aa, Artificial Sequence
  anti-microbial peptide
WLKKIGKKIERVGQHTRDATIQGLGIAQQAANVAATAR

SEQ ID NO: 52 - protein, 13 aa, Artificial Sequence
  anti-microbial peptide
GTNNWWQSPSIQN